(19) United States
(12) Patent Application Publication
(10) Pub. No.: US 2006/0089515 A1
(43) Pub. Date: Apr. 27, 2006

(54) Process for continuously preparing alkylene glycol diethers

(75) Inventors: Achim Stankowiak, Altoetting (DE); Sibylle Schwarz, Mettenheim (DE); Oliver Pfueller, Sulzbach (DE); Alexander Snell, Altoetting (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant GmbH

(21) Appl. No.: 11/256,126

(22) Filed: Oct. 21, 2005

(30) Foreign Application Priority Data
Oct. 21, 2004 (DE) 10 2004 051 267.1

(51) Int. Cl. C07C 41/00

(52) U.S. Cl. 568/579

(57) ABSTRACT

The invention provides a process for preparing alkylene glycol diethers of the formula (I) 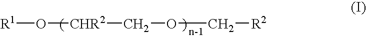
by reacting compounds of the formula (II) 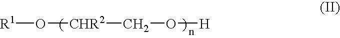
in which R¹ is hydrogen or C₁- to C₃-alkyl, R² is hydrogen, CH₃ or CH₂CH₃, and n is from 1 to 4, in the liquid phase in the presence of catalysts at temperatures between 170 and 300° C., and continuously distilling off the compounds of the formula (I).

The present invention relates to a continuous process for preparing catenated alkylene glycol diethers.

Alkylene glycol diethers have been used for a long time as polar, aprotic, inert solvents. Alkylene glycol diethers find use in particular as high-boiling solvents in the pharmaceutical and chemical industry, but also as binders in coatings or as solvents in detergent formulations.

For their preparation, what are called indirect processes, for example the Williamson ether synthesis (K. Weissermel, H. J. Arpe “Industrielle Organische Chemie” [Industrial organic chemistry], 1998, page 179) or the hydrogenation of diglycol ether formal (DE-A-24 34 057), are employed industrially or described. However, both processes have disadvantages: the two-stage Williamson ether synthesis has low economic viability by virtue of the stoichiometric consumption of chlorine and alkali, and also the removal of the water of reaction and sodium chloride which forms. The hydrogenation of formal is carried out under high pressure, which has the prerequisite of high capital costs in the plant construction and is therefore unsuitable for smaller production amounts.

In what are called direct processes, alkylene oxide is inserted into a catenated ether in the presence of Lewis acids such as BF₃ (U.S. Pat. No. 4,146,736 and DE-A-26 40 505 in conjunction with DE-A-31 28 962) or SnCl₄ (DE-A-30 25 434). The disadvantage of these processes is that large amounts of cyclic by-products, for example dioxane or dioxolane, are unavoidably formed.

An alternative synthesis means is the catalytic dehydrodecarbonylation of glycols and methylglycols:

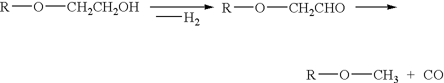

DE-A-29 00 279 describes this synthetic route by the reaction of polyethylene glycols or polyethylene glycol monomethyl ethers in the gas phase at 250-500° C. in the presence of supported palladium, platinum, rhodium, ruthenium or iridium catalysts and hydrogen.

JP-A-600 28 429 describes the reaction of C₄ and longer-chain monoalkyl ethers using a nickel/rhenium catalyst supported on γ-alumina. In this process too, hydrogen is supplied. Likewise known is the hydrogenation of secondary hydroxyl groups with hydrogen at standard pressure using supported nickel catalysts (DE-A-38 02 783). In this process, the synthesis explicitly does not succeed when Raney nickel is used.

U.S. Pat. No. 3,428,692 discloses that it is possible by heating C₆- to C₁₂-chain monoalkyl and monophenyl ethers to 200-300° C. in the presence of nickel and cobalt catalysts to prepare the corresponding deformylated methyl-capped ethoxylates. However, this forms mixtures of the desired methyl ethers with ethoxylates which have not been fully converted and from 20 to 30% of unidentified aldehyde compounds. EP-A-00 43 420 describes a similar process using palladium, platinum or rhodium catalysts supported on Al₂O₃ or SiO₂.

All processes described in the current prior art either have low selectivity or else are technically very complex and therefore uneconomic for the preparation of short-chain alkylene glycol diethers. The object arising therefrom has been achieved in accordance with the invention according to the specifications of the claims.

Surprisingly, it is possible to react short-chain alkylene glycols and alkylene glycol monoethers in a simple continuous process by catalysis to give the desired alkylene glycol diethers.

The invention thus provides a continuous process for preparing alkylene glycol diethers of the formula (I) 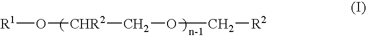
by reacting compounds of the formula (II) 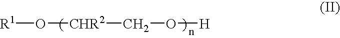
in which R¹ is hydrogen or C₁- to C₃-alkyl, R² is hydrogen, CH₃ or CH₂CH₃, and n is from 1 to 4, in the liquid phase in the presence of catalysts at temperatures between 170 and 300° C., and continuously distilling off the compounds of the formula (I).

Suitable starting materials are either pure ethoxylates, propoxylates or butoxylates, or the mixed alkoxylates formed from ethylene oxide, propylene oxide and/or butylene oxide, and also monoalkyl alkoxylates based on ethylene oxide, propylene oxide and/or butylene oxide. R¹ is preferably H or methyl.

R² is preferably hydrogen and n is preferably from 1 to 3. Particular preference is given to using methyldiglycol and methyltriglycol. The starting material of the formula II is fed to the reaction continuously, while the product which forms is simultaneously removed continuously, for example by distillation.

Suitable catalysts are preferably pure nickel catalysts, and also mixtures of nickel (including Raney nickel) with other metals, for example palladium, platinum, cobalt, rhodium, iridium, iron, ruthenium, osmium, manganese, rhenium, chromium, molybdenum, copper (including Raney copper) or bismuth. These mixtures may either be of heterogeneous nature or be dopants on the catalyst surface. The support material is not critical. Suitable support materials are, for example, alumina and other metal oxides, carbon, kieselguhr, silicon dioxide, silicon carbide, zeolites and the like. Preference is given to using a mixture of Raney nickel with palladium on activated carbon. The amount of catalyst to be used can vary within wide limits. In general, sufficient catalyst is used that from 0.2 to 15% by weight of catalyst based on the use amount of the compound of the formula (I), preferably from 0.5 to 10% by weight of catalyst based on the use amount of the compound of the formula (I), is present.

The reaction over the catalysts is effected preferably at from 180 to 250° C. The reaction is generally carried out at standard pressure, but it is also possible to work under reduced or elevated pressure. In the process according to the invention, hydrogen or an inert gas may be used as a carrier gas.

The process according to the invention will now be illustrated in detail with reference to some examples:

### EXAMPLE 1

### Preparation of monoethylene glycol dimethyl ether

A 10 liter stirred autoclave equipped with heatable vapor tube was initially charged with 5.0 kg of methyldiglycol (41.7 mol), 624 g of Pd catalyst (5% on activated carbon) and 150 g of Raney nickel. The mixture was heated gradually to 220° C. at an elevated pressure of 3.0 bar and the products which formed were distilled off continuously via the vapor tube. At the same time, methyldiglycol was metered in continuously, so that the fill level in the reactor remained approximately constant. After the distillation via the vapor tube, a product mixture consisting of 75% monoethylene glycol dimethyl ether, 11% diethylene glycol dimethyl ether, 2% triethylene glycol dimethyl ether, 10% methylglycol and 2% further by-products was obtained. A second distillation through a column (18 theoretical plates, reflux ratio 1:3) separates these products from one another and methyidiglycol is recycled. The experiment is ended after 10 hours.

The yield of monoethylene glycol dimethyl ether was 995.8 g/h.

### EXAMPLE 2

### Preparation of diethylene glycol dimethyl ether

Process analogous to example 1 with the following changes:

5.0 kg of methyltriglycol (30.5 mol), 624 g of Pd catalyst (5% on activated carbon) and 150 g of Raney nickel.

The product mixture consisted of 74% diethylene glycol dimethyl ether, 20% methyltriglycol, 4% methyldiglycol and 2% further by-products.

The yield of diethylene glycol dimethyl ether was 1839.6 g/h.

### Example 3

### Preparation of diethylene glycol dimethyl ether

A 250 ml reaction flask equipped with stirrer, baffles, dropping funnel and distillation head with condenser was initially charged with 180.2 g (1.1 mol) of methyltriglycol, 9.7 g of palladium catalyst (5% m/m palladium on activated carbon) and 3.7 g of Raney nickel catalyst. The initially charged mixture was heated to 220° C. with stirring. The products which formed were distilled off continuously via the distillation head with condenser. In parallel, the methyltriglycol reactant was metered in continuously via a dropping funnel, in such a way that the ratio of methyltriglycol added dropwise to product distilled off remained constant.

The product mixture consisted of 67% diethylene glycol dimethyl ether, 25% methyltriglycol, 5% methyidiglycol and 3% further by-products.

The yield of diethylene glycol dimethyl ether was 70.8 g/h.

### EXAMPLE 4

### Preparation of diethylene glycol dimethyl ether

Process analogous to example 3 with the following changes:

180.5 g (1.1 mol) of methyltriglycol and 2.8 g of Raney nickel catalyst.

The product mixture consisted of 52% diethylene glycol dimethyl ether, 20% methyltriglycol, 21% methyldiglycol and 7% further by-products.

The yield of diethylene glycol dimethyl ether was 33.1 g/h.

### EXAMPLE 5

### Preparation of diethylene glycol dimethyl ether

Process analogous to example 3 with the following changes:

182.6 g (1.12 mol) of methyltriglycol and 3.4 g of Raney nickel catalyst and 3.4 g of Raney copper catalyst.

The product mixture consisted of 56% diethylene glycol dimethyl ether, 13% methyltriglycol, 20% methyidiglycol and 11% further by-products.

The yield of diethylene glycol dimethyl ether was 25.2 g/h.

### EXAMPLE 6

### Preparation of diethylene glycol dimethyl ether

Process analogous to example 3 with the following changes:

180.6 g (1.1 mol) of methyltriglycol and 1.5 g of Raney nickel, 2.7 g of palladium and 0.9 g of rhodium.

The product mixture consisted of 68% diethylene glycol dimethyl ether, 17% methyltriglycol, 10% methyldiglycol and 5% further by-products.

The yield of diethylene glycol dimethyl ether was 25.1 g/h.

### EXAMPLE 7

### Preparation of diethylene glycol dimethyl ether

Process analogous to example 3 with the following changes:

180.1 g (1.1 mol) of methyltriglycol and 1.4 g of Raney nickel, 2.7 g of palladium and 0.9 g of rhenium.

The product mixture consisted of 63% diethylene glycol dimethyl ether, 10% methyltriglycol, 16% methyldiglycol and 11% further by-products.

The yield of diethylene glycol dimethyl ether was 20.1 g/h.

### EXAMPLE 8

### Preparation of diethylene glycol dimethyl ether

Process analogous to example 3 with the following changes:

181.1 g (1.1 mol) of methyltriglycol and 1.9 g of Raney nickel and 2.5 g of platinum doped with bismuth.

The product mixture consisted of 54% diethylene glycol dimethyl ether, 16% methyltriglycol, 21% methyidiglycol and 9% further by-products.

The yield of diethylene glycol dimethyl ether was 26.6 g/h.

1. A continuous process for preparing alkylene glycol diethers of the formula (I) 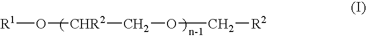
by reacting compounds of the formula (II) 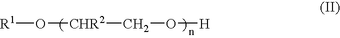
in which R¹ is hydrogen or C₁- to C₃-alkyl, R² is hydrogen, CH₃ or CH₂CH₃, and n is from 1 to 4, in a liquid phase in the presence of a catalyst at a temperature between 170 and 300° C., and continuously distilling off the compounds of the formula (I).

2. The process as claimed in claim 1, in which R¹ is H or methyl.

3. The process as claimed in claim 1, in which R² is H.

4. The process of claim 1, in which n is from 1 to 3.

5. The process of claim 1, in which the catalyst comprises nickel.

6. The process of claim 1, in which the catalyst is a pure nickel catalyst.

7. The process of claim 1, in which the catalyst comprises, in addition to nickel, one or more metals selected from the group consisting of Pd, Pt, Co, Rh, Ir, Fe, Ru, Os, Mn, Re, Cr, Mo, Cu, Bi, and mixtures thereof.

8. The process of claim 1, in which the catalyst is present in an amount of from 0.2 to 15% by weight based on the amount of the compound of the formula (I).

9. A process for the continuous production of alkylene glycol diethers of formula (1) 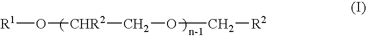
said process comprising:
a) contacting a liquid feed stream comprising a compound of formula (II): 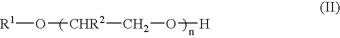
in which R¹ is hydrogen or C₁- to C₃-alkyl, R² is hydrogen, CH₃ or CH₂CH₃, and n is from 1 to 4, with a catalyst comprising nickel at a temperature between 170 and 300° C. to produce a reaction product stream comprising the compound of formula (I) and the compound of formula (II);
b) continuously separating the reaction product stream to provide a finished product stream comprising the compounds of the formula (I) and a recycle stream comprising the compound of formula (II); and
c) recycling at least a portion of the recycle stream to step (a).

10. The process of claim 9, wherein step (b) comprises distillation.

11. The process of claim 9, wherein said catalyst comprises pure nickel.

12. The process of claim 9, wherein said catalyst comprises a metal selected from the group consisting of nickel, Raney nickel, palladium, platinum, colbalt, rhodium, iridium, iron, ruthenium, osmium, manganesem rhenium, chromium, molybdenum, copper, Raney copper, bismuth and mixtures thereof.
